(19) United States
(12) Patent Application Publication
(10) Pub. No.: US 2021/0289032 A1
(43) Pub. Date: Sep. 16, 2021

(54) SURGICAL TABLE DATA TRANSMISSION SYSTEM

(71) Applicant: TRUMPF MEDIZIN SYSTEME GMBH + CO. KG, Saalfeld (DE)

(72) Inventors: Jason S. BERNOTSKY, Dunmore, PA (US); Michael C. PIERCE, Harvard, MA (US); Phillip B. DOLLIVER, Framingham, MA (US)

(21) Appl. No.: 17/196,122

(22) Filed: Mar. 9, 2021

(30) Foreign Application Priority Data
Mar. 10, 2020 (EP) 20162153.9

(51) Int. Cl. H04L 29/08; H04L 12/66; G16H 40/67; A61G 13/10

(52) U.S. Cl. CPC H04L 67/12; H04L 12/66; G16H 40/67; A61G 13/10; A61G 2203/44; A61G 2203/42; A61G 2203/46; A61G 2203/12; A61G 2203/34

(57) ABSTRACT

A surgical table data transmission system is provided. The system comprises a data aggregation node, and at least one remote node attachable to a component or an accessory of a surgical table, wherein the data aggregation node and the remote node are configured to exchange data by a first wired or wireless communication, the data aggregation node is configured to exchange the data with at least one of a controller of the surgical table and a hospital network gateway by a second wired or wireless communication, and the remote node is configured to transmit identification data of the component or accessory or to transmit received sensor data to the data aggregation node.

### CROSS-REFERENCE TO RELATED APPLICATIONS

This application claims the benefit of European Patent Application Serial No. EP20162153.9, filed Mar. 10, 2020, the entire disclosure of which is incorporated herein by reference in its entirety.

### BACKGROUND

The present disclosure relates to a surgical table data transmission system, and in particular, to a surgical table data transmission system using a data network.

Currently, beyond a basic presence detection, the majority of surgical tables do not have any smart sensing or control features integrated with removable components or accessories. The lack of these capabilities limits the ability of the surgical tables to lock out certain unsafe movements.

Moreover, due to various combinations, a multitude of table components and accessories require a more complex control for the table which is accompanied by extensive wiring and configuration of controllers being required.

The object underlying the present disclosure is to remedy these disadvantages and to provide a surgical table data transmission system, a component of a surgical table, an accessory of the surgical table, and a surgical table enabling a flexible data exchange between a data processing device and components of the surgical table.

### SUMMARY

The present disclosure includes one or more of the features recited in the appended claims and/or the following features which, alone or in any combination, may comprise patentable subject matter.

According to an aspect of the present disclosure, a surgical table data transmission system comprises at least one remote node attachable to a component or an accessory of a surgical table and a data aggregation node. The remote node and the data aggregation node are configured to be able to exchange data with wired or wireless communication. The remote node is configured to transmit identification data of the component or accessory or to transmit received sensor data to the data aggregation node, and the data aggregation node is configured to exchange the data with at least one of a controller of the surgical table and a hospital network gateway by a second wired or wireless communication connection.

The nodes are to be understood as network nodes hardware which can receive, create, store or send data along distributed network routes.

By the data transmission system, data, such as identifiers, e.g., stored identification data, received sensor data or inputs by an operator, can be transmitted from several components or accessories of the surgical table via an assigned remote node which can be attached to the component or accessory and the data aggregation node to the controller of the surgical table or to the hospital network gateway. On the other hand, control signals from the controller of the surgical table or information of the hospital network can be transmitted via the data aggregation node and the remote node to actors or displays on the components or accessories of the surgical table. Therefore, a transmission of data can be performed without the necessity of individual data lines which enables a lean structure of the data transfer and a flexibility in case of a change of the configuration of the surgical table.

The data can be transmitted wirelessly or in a wired manner. In case of the wired manner, the principle of plug and receptacle is used or contacts which automatically establish a contact when attaching the component or the accessory to the surgical table are provided. In this case, for example, a RS-485 or a CAN protocol is used.

In an embodiment of the surgical table data transmission system, the data aggregation node and the remote node respectively comprise at least one of a transmitter and a receiver for radio communication between the data aggregation node and the remote node.

Due to this configuration, the wireless communication between the data aggregation node and the remote node is possible. Therefore, no wiring is necessary so that the effort for wiring can be saved and, also, there is no risk for damaging a cable. Therefore, safety of the operation of the surgical table is enhanced. As transmission protocols for the communication, for example, Zigbee, Near Field Communication (NFC), Bluetooth or WLAN can be used.

In another embodiment of the surgical table data transmission system, the data aggregation node comprises at least one of a transmitter and a receiver for radio communication with the controller of the surgical table or the hospital network gateway.

By this configuration, the wireless communication between the data aggregation node and the controller of the surgical table or the hospital network gateway is possible. Therefore, no wiring is necessary so that the effort for wiring can be saved and, also, there is no risk for damaging a cable. Therefore, safety of the operation of the surgical table is enhanced. As a transmission protocol for the communication, for example, WLAN can be used.

According to a further aspect of the present disclosure, a component of a surgical table is provided, wherein the component comprises the remote node of the surgical table data transmission system.

By providing the component of the surgical table with the remote node, specific information concerning the component can be provided by the remote node and, as the case may be, functions of the component, such as motions by a controlled drive, can be performed by being controlled via the remote node.

In an embodiment of the component, the component comprises a memory device configured to store the identification data of the component.

By the identification data of the component stored in the memory device thereof, the controller of the surgical table can recognize the type of the component and can adjust control signals in order to, e.g., avoid collisions with other components of the surgical table or with the floor. Also, an automatic recognition of the component when being attached to the surgical table is possible.

In another embodiment of the component, the component comprises a sensor configured to transmit sensor data to the remote node.

Due to the component comprising the sensor, for example, monitoring a patient lying on the component of the surgical table is possible. Furthermore, monitoring the component for avoiding damage, e.g., by overload or collision, is possible. By the transmission of the sensor signals via the remote node and the data aggregation node, no further signal path to the controller of the surgical table is necessary so that additional efforts can be saved.

In another embodiment of the component, the sensor is at least one of a pressure mapping sensor, a temperature sensor, a patient weight sensor, an angle measuring sensor, and a collision detection sensor.

By deploying the pressure mapping sensor, the temperature sensor and/or the patient weight sensor, the patient lying on the component of the surgical table can be monitored in order to, e.g., avoid decubitus ulcers or hypothermia during a surgical intervention. By the use of the angle measuring sensor and/or the collision detection sensor, the patient and the surgical staff can be kept safe since inadmissible states of the surgical table can be determined.

In a yet another embodiment of the component, the component comprises a battery configured to supply energy to the remote node.

When providing the component with a battery, no line for supplying energy to the remote node or, as the case may be, the sensor is necessary. Therefore, for this purpose, no wiring is necessary so that this effort can be saved and, also, there is no risk for damaging a cable which enhances safety of the operation of the surgical table.

According to a further aspect of the present disclosure, an accessory of a surgical table is provided, wherein the accessory comprises the remote node of the surgical table data transmission system.

By providing the accessory of the surgical table with the remote node, an automatic recognition of the accessory when being attached to the surgical table is possible. Further, specific information of the accessory can be provided by the remote node and, as the case may be, functions of the accessory, such as motions executed by a controlled drive, can performed by being controlled via the remote node.

In an embodiment of the accessory, the accessory comprises a memory device configured to store the identification data of the accessory.

By the identification data of the accessory stored in the memory device thereof, for example, the controller of the surgical table can recognize the type of the accessory and can adjust control signals in order to, e.g., avoid collisions with components of the surgical table or with the floor.

In a further embodiment of the accessory, the accessory comprises a sensor configured to transmit sensor data to the remote node.

Due to the accessory comprising the sensor, for example, monitoring a patient being in contact with the accessory of the surgical table is possible. Furthermore, monitoring the accessory for avoiding damage, e.g., by overload or collision, is possible. By the transmission of the sensor signals to the remote node, no further signal path to the controller of the surgical table is necessary.

In a further embodiment of the accessory, the accessory comprises a battery configured to supply energy to the remote node.

When providing the accessory with a battery, no line for supplying energy to the remote node or the sensor is necessary. Therefore, no wiring therefore is necessary so that this effort can be saved and, also, there is no risk for damaging a cable which enhances safety of the operation of the surgical table.

According to a further aspect of the present disclosure, a surgical table comprises a surgical table data transmission system, at least one of a component and an accessory, and a controller of the surgical table, wherein the controller is configured to control functions of the surgical table. The data aggregation node is configured to exchange the system data with the controller.

In the surgical table equipped in this manner, the data can be transmitted, if provided, from several components or accessories to the controller of the surgical table via a remote node assigned to the component or the accessory and which can be attached to the component or the accessory and the data aggregation node. On the other hand, control signals or information of the controller can be transmitted via the data aggregation node and the remote node to actors or displays on the components or accessories of the surgical table. Therefore, the transmission of data can be performed without the necessity of individual data lines which enables a lean structure of the data transfer and a flexibility in case of a change of the configuration of the surgical table.

According to an embodiment of the surgical table, the data aggregation node is integrated in the controller of the surgical table.

By the integration of the data aggregation node in the controller of the surgical table, no further device, as the case may be, comprising a separate casing, is necessary in order to avoid additional efforts.

According to another embodiment of the surgical table, the data aggregation node is a separate device connected to the controller of the surgical table.

When providing the data aggregation node as a separate device, for example as an upgrade device or dongle, an upgrade of the functionality of the surgical table is possible without big additional efforts.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 shows a surgical table according to the present disclosure;

FIG. 2 shows a surgical table component identification and sensing system connection diagram; and

FIG. 3 shows a remote identification/sensing node basic electrical diagram.

### DETAILED DESCRIPTION

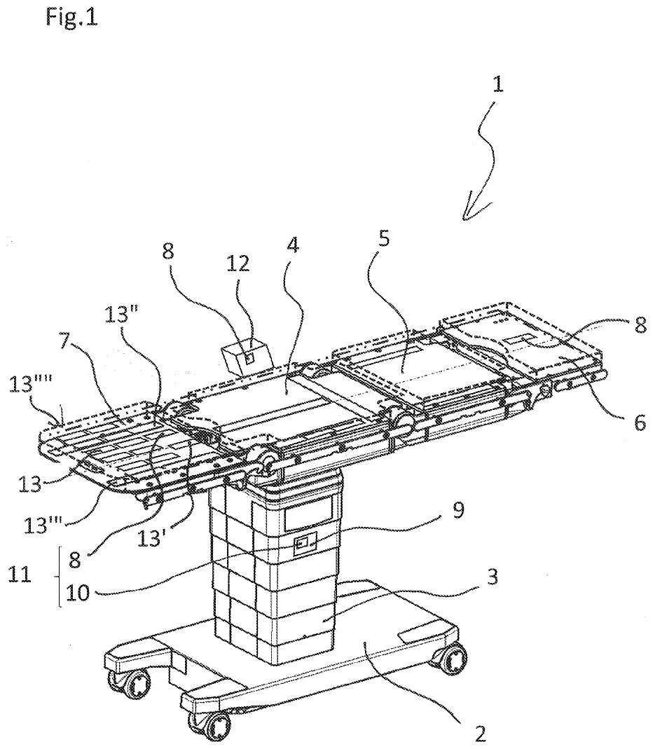

FIG. 1 shows a surgical table 1 according to the present disclosure. The surgical table 1 comprises a base 2, a column 3, a seat plate 4 and a back plate 5. Nevertheless, the surgical table 1 can alternatively have another structure.

Further, the surgical table 1 comprises a head plate 6 and a leg plate 7 as components which are detachable. In alternative embodiments, merely some of these components are provided, not all of these components are detachable, no detachable component is provided or other or additional components are provided.

The detachable components 6, 7 are respectively provided with a remote identification/sensing node as a remote node 8. In alternative embodiments, not all of the detachable components 6, 7 are provided with a remote node 8 or components of the surgical table 1 which are not detachable, such as the seat plate 4 or the back plate 5, are also provided with the remote node 8.

In the shown embodiment, the leg plate 7 is provided with a pressure mapping sensor 13 configured to detect a pressure distribution on a surface of the leg plate 7. Further, the leg plate 7 is provided with a temperature sensor 13′ for measuring a body temperature of a patient lying on the tabletop, a patient weight sensor 13″, an angle measurement sensor 13′″ for measuring an inclination of the component 7, and a collision detection sensor 13″″ detecting a collision between the component 7 and another component of the surgical table 1 or the environments of the surgical table 1. In alternative embodiments, the detachable components 6, 7 comprise other sensors, all of the components 4, 5, 6, 7 are respectively provided with at least one sensor 13, 13′, 13″, 13″, 13″″, or none of the components 4, 5, 6, 7 is provided with a sensor.

The surgical table 1 further comprises an accessory 12 which is illustrated in a principal manner. The accessory 12 is a pad with pressure sensing. Alternatively, the accessory 12 can be an electrically adjustable support device. The accessory 12 is also provided with one of the remote nodes 8. In alternative embodiments, several accessories 12 having a remote node 8 are provided, no accessory 12 is provided or not all of the accessories 12 are provided with the remote node 8.

The surgical table 1 comprises a controller 9 which is configured to control functions of the surgical table 1. In particular, the controller 9 controls motions of the surgical table 1, such as a height adjustment of a tabletop comprising the seat plate 4, the back plate 5, the head plate 6 and the leg plate 7. The controller 9 further controls tilting of the tabletop around its longitudinal axis and tilting of the tabletop around an axis perpendicular to the longitudinal axis into a Trendelenburg or Anti-Trendelenburg position.

The controller 9 comprises an integrated central control node as data aggregation node 10. The data aggregation node 10 exchanges data with the controller 9. Alternatively, the data aggregation node 10 is a separate device connected to the controller 9 of the surgical table. Such a separate device is, for example, an upgrade device or a dongle. Also, alternatively, the separate data aggregation node 10 can be provided in a wall control unit, a surgical light, etc.

In an embodiment in which the non-detachable components 4, 5 are provided with sensors, these sensors are either directly connected to the controller 9 or connected to the controller 9 also via the remote node 8 and the data aggregation node 10.

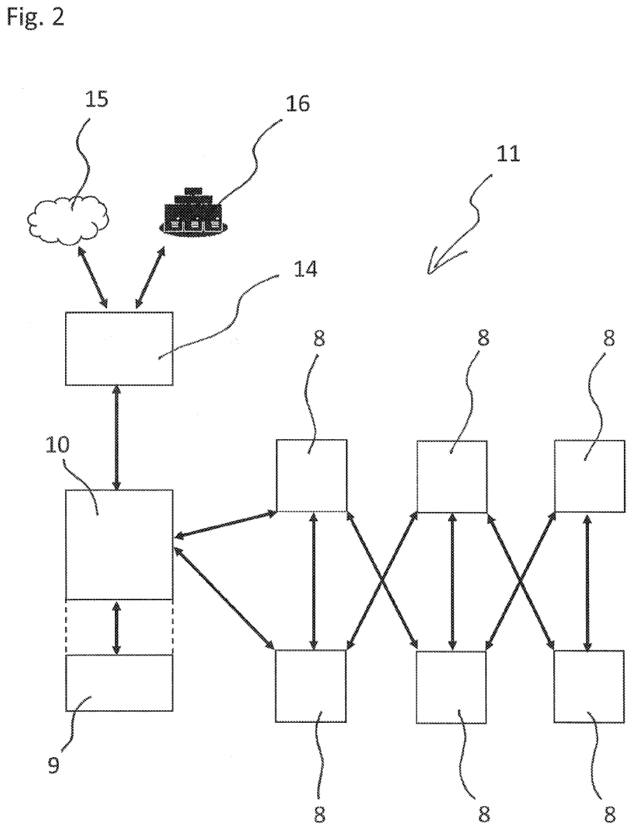

FIG. 2 shows a surgical table component identification and sensing system connection diagram.

The surgical table component identification and sensing system connection diagram shows a surgical table data transmission system 11 comprising the data aggregation node 10 and the remote nodes 8. As mentioned above, the data aggregation node 10 is integrated in the controller 9 of the surgical table 1 and the remote nodes 8 are attachable to the components 6, 7.

The data aggregation node 10 and the remote nodes 8 exchange data by a wireless communication. The remote nodes 8 also exchange data among each other. Thereto, the data aggregation node 10 and the remote nodes 8 respectively comprise a transmitter and/or a receiver for radio communication. In this embodiment, the data exchange is performed via WLAN. Alternatively, “Zigbee”, “Near field Communication” or “Bluetooth” can be used for the wireless communication or a CAN or a RS-485 protocol can be used for a wired communication.

The data aggregation node 10, on the one hand, exchanges system data with the controller 9 of the surgical table 1 and/or, on the other hand, with a hospital network gate way 14. The hospital network gateway 14 is connected to a hospital cloud 15 and/or a hospital data network 16. The data are either directly forwarded from one of the remote nodes 8 or processed by the data aggregation node 10 or the controller 9 of the surgical table 1. The data exchange between the data aggregation node 10 and the hospital network gateway 14 is performed by a wired communication via the RS-485 protocol. Alternatively, another protocol, such as CAN, or a wireless communication, e.g., via WLAN, is used. Then, the data aggregation node 10 comprises at least one of a transmitter and a receiver for radio communication to the controller 9 of the surgical table 1 or the hospital network gateway 14.

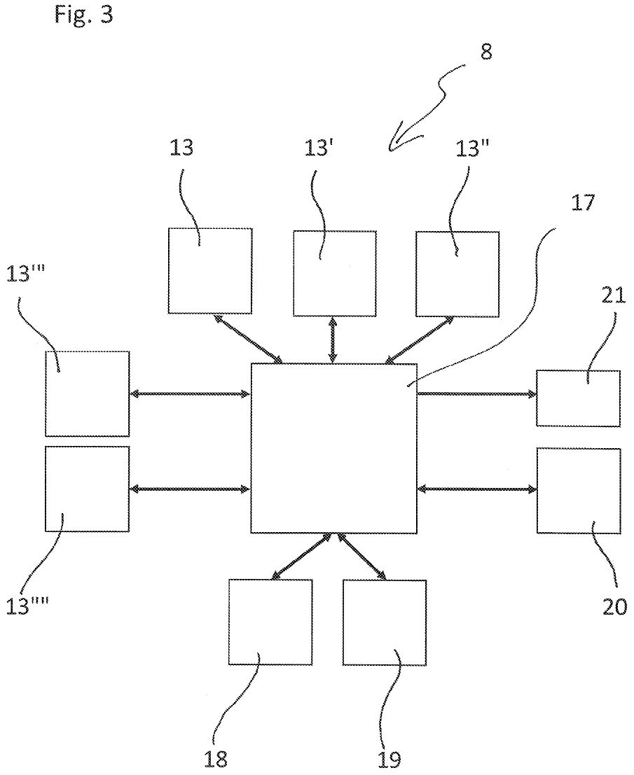

FIG. 3 shows a remote identification/sensing node basic electrical diagram of the remote node 8.

FIG. 3 shows that a microcontroller 17 is connected to the pressure mapping sensor 13, the temperature sensor 13′, the patient weight sensor 13″, the angle measurement sensor 13′″, and the collision detection sensor 13″″. The sensors 13, 13′, 13″, 13′″, 13″″ are provided in/on the components 6, 7 or the accessory 12. The sensors 13, 13′, 13″, 13′″, 13″″ transmit sensor data to the remote node 8. As mentioned above, alternatively, not all of these sensors are provided, or some of these sensors or other sensors are provided in/on the components 6, 7 or the accessory 12.

The microcontroller 17 is further connected to a battery 18 configured to supply energy to the remote node 8. The battery 18 is provided in/on the component 6, 7 and/or the accessory 12 for supplying the remote node 8 and, as the case may be, the sensors with energy. Alternatively, no battery 18 is provided and the remote node 8 is, for example, supplied with energy by wiring.

Furthermore, the microcontroller 17 is connected to a wireless interface 19. The interface 19 serves for the data exchange with another remote nodes 8 or with the data aggregation node 10 as already mentioned above.

Moreover, the microcontroller 17 is connected to a memory device 20 which stores the identification data of the component 6, 7 and/or of the accessory 12. The component 6, 7 and/or the accessory 12 comprises the memory device 20. Alternatively, the identification data are not provided or stored in another manner, e.g., by a specific shape of a mechanical interface.

The microcontroller 17 is further connected to a simple local indicator 21 which serves as a user interface. Alternatively, no such simple local indicator 21 is provided on the component 5, 6 and the accessory 12.

In use, when starting up the controller 9 of the surgical table 1 or when attaching one of the components 6, 7 or the accessory 12 to the surgical table 1, the remote node 8 transmits data for a component or accessory type identification to the data aggregation node 10. Alternatively, this data can also be transmitted several times, e.g., cyclically. By this data, a configuration of the surgical table 1 can be determined and correct accessories 12 and components 6, 7 can be planned to be in place by hospital staff for a planned surgical procedure.

Furthermore, the remote node 8 transmits senor data of the sensors 13, 13′, 13″, 13′″, 13″″ to the data aggregation node 10. The sensor data are transmitted cyclically. This sensor data can be used to keep patient and surgical staff safe. Alternatively, the sensor data are not transmitted cyclically but only upon change of the sensor data and if, for example, no sensors are provided, no sensor data are transmitted.

The data is transmitted to the controller 9 of the surgical table 1 for appropriately controlling the surgical table 1. Furthermore, alternatively or additionally, the data is transmitted to the hospital network gateway 14 where it is forwarded to hospital staff for planning purposes.

While the present disclosure has been illustrated and described in detail in the drawing and the foregoing description, such illustration and description are to be considered illustrative or exemplary and not restrictive. The invention is not limited to the disclosed embodiments. From reading the present disclosure, other modifications will be apparent to a person skilled in the art. Such modifications may involve other features, which are already known in the art and may be used instead of or in addition to features already described herein. In the claims, the word “comprising” does not exclude other elements or steps, and the indefinite article “a” or “an” does not exclude a plurality.

Although this disclosure refers to specific embodiments, it will be understood by those skilled in the art that various changes in form and detail may be made without departing from the subject matter set forth in the accompanying claims.

1. A surgical table data transmission system comprising
at least one remote node attachable to a component or an accessory of a surgical table, and
a data aggregation node, wherein
the remote node and the data aggregation node are configured to exchange data by a first wired or wireless communication,
the remote node is configured to transmit identification data of the component or accessory or to transmit received sensor data to the data aggregation node, and
the data aggregation node is configured to be able to exchange the data with at least one of a controller of the surgical table and a hospital network gateway by a second wired or wireless communication.

2. The surgical table data transmission system of claim 1, wherein the data aggregation node and the remote node respectively comprise at least one of a transmitter and a receiver for radio communication between the data aggregation node and the remote node.

3. The surgical table data transmission system of claim 1, wherein the data aggregation node comprises at least one of a transmitter and a receiver for radio communication with the controller of the surgical table or the hospital network gateway.

4. The surgical table data transmission system of claim 1, further comprising a component, wherein the component comprises a remote node of the surgical table data transmission system.

5. The surgical table data transmission system of claim 4, wherein the component comprises a memory device configured to store the identification data of the component.

6. The surgical table data transmission system of claim 4, wherein the component comprises a sensor configured to transmit sensor data to the remote node.

7. The surgical table data transmission system of claim 6, wherein the sensor is at least one of a pressure mapping sensor, a temperature sensor, a patient weight sensor, an angle measuring sensor, and a collision detection sensor.

8. The surgical table data transmission system of claim 4, wherein the component comprises a battery configured to supply energy to the remote node.

9. The surgical table data transmission system of claim 1, further comprising an accessory, wherein the accessory comprises a remote node of the surgical table data transmission system.

10. The surgical table data transmission system of claim 9, wherein the accessory comprises a memory device configured to store the identification data of the accessory.

11. The surgical table data transmission system of claim 9, wherein the accessory comprises a sensor configured to transmit sensor data to the remote node.

12. The surgical table data transmission system of claim 9, wherein the accessory comprises a battery configured to supply energy to the remote node.

13. A surgical table comprising
a surgical table data transmission system including
at least one remote node attachable to a component or an accessory of a surgical table, and
a data aggregation node, wherein
the remote node and the data aggregation node are configured to exchange data by a first wired or wireless communication,
the remote node is configured to transmit identification data of the component or accessory or to transmit received sensor data to the data aggregation node, and
the data aggregation node is configured to be able to exchange the data with at least one of a controller of the surgical table and a hospital network gateway by a second wired or wireless communication,
a component,
an accessory, and
a controller of the surgical table, the controller being configured to control functions of the surgical table,
wherein the data aggregation node is configured to exchange the data with the controller.

14. The surgical table of claim 13, wherein the data aggregation node is integrated in the controller of the surgical table.

15. The surgical table of claim 13, wherein the data aggregation node is a separate device connected to the controller of the surgical table.

16. The surgical table of claim 13, wherein the accessory comprises a memory device configured to store the identification data of the accessory.

17. The surgical table of claim 13, wherein the accessory comprises a sensor configured to transmit sensor data to the remote node.

18. The surgical table of claim 13, wherein the accessory comprises a battery configured to supply energy to the remote node.

19. The surgical table of claim 13, wherein the component comprises a sensor configured to transmit sensor data to the remote node.

20. The surgical table of claim 19, wherein the sensor is at least one of a pressure mapping sensor, a temperature sensor, a patient weight sensor, an angle measuring sensor, and a collision detection sensor.
